(19) United States
(12) Patent Application Publication
(10) Pub. No.: US 2024/0197582 A1
(43) Pub. Date: Jun. 20, 2024

(54) HEXAGONAL BORON NITRIDE POWDER FOR COSMETICS, AND COSMETIC

(71) Applicant: Denka Company Limited, Tokyo (JP)

(72) Inventor: Ryuki MATSUI, Tokyo (JP)

(73) Assignee: Denka Company Limited, Tokyo (JP)

(21) Appl. No.: 18/287,235

(22) PCT Filed: Mar. 22, 2022
(86) PCT No.: PCT/JP2022/013254

§ 371 Date: Oct. 17, 2023

(30) Foreign Application Priority Data
Apr. 19, 2021 (JP) 2021-070290

(51) Int. Cl. A61K 8/19; A61K 8/02; A61Q 1/12

(52) U.S. Cl. CPC A61K 8/19; A61K 8/0245; A61Q 1/12

(57) ABSTRACT

A hexagonal boron nitride powder for cosmetics containing primary particles of hexagonal boron nitride, in which an aspect ratio of the primary particles is 25 or less, and an oil absorption value is 50 to 90 mL/100 g.

### TECHNICAL FIELD

The present disclosure relates to a hexagonal boron nitride powder for cosmetics, and a cosmetic.

### BACKGROUND ART

Hexagonal boron nitride has lubricity, high heat conductivity, and insulation properties, and is used for various purposes such as solid lubricants, mold release materials, fillers for resins and rubber, raw materials for cosmetics, and insulating sintered bodies having heat resistance. A hexagonal boron nitride powder has the function of improving the smoothness, the spreadability, the concealability, and the like of cosmetics, and the function of imparting glowing properties and the like to cosmetics.

As extender pigments that can exhibit the same functions as those of hexagonal boron nitride powder, talc powder, mica powder, and the like are used. However, because natural minerals are used in talc powder and the like, there are large variations in particle size and thickness, and additional adjustment is required to produce cosmetics with stable quality. The hexagonal boron nitride powder can be adjusted in regard to particle size, thickness, and the like, and thus is excellent in smoothness as compared to talc powder and mica powder. Accordingly, the hexagonal boron nitride powder is often used in cosmetics that require excellent smoothness. Patent Literature 1 proposes a hexagonal boron nitride powder in which the ratio of shear stress to pressing force is set within a predetermined numerical value range to improve smoothness.

The characteristics required for cosmetics are wide-ranging, and the level of the characteristics required for a hexagonal boron nitride powder as a raw material are also becoming higher. For example, Patent Literature 2 proposes a hexagonal boron nitride powder with reduced hydrophilic functional groups on the surface and an increased oil absorption value as a hexagonal boron nitride powder for cosmetics which is excellent in film formation properties in thin films and has improved cooling sensation and transparency (no-makeup makeup look).

### CITATION LIST

### Patent Literature

[Patent Literature 1] Japanese Unexamined Patent Publication No. 2019-043792
[Patent Literature 2] Japanese Unexamined Patent Publication No. 2014-094878

### SUMMARY OF INVENTION

### Technical Problem

Meanwhile, in order to improve spreadability, which is one of feelings during use of cosmetics, it is conceivable to reduce the particle size of the primary particles of hexagonal boron nitride as an extender pigment. However, when the particle size is reduced, a cosmetic containing a hexagonal boron nitride powder may appear whitened by light scattering or the like, which may impair the glossiness of a makeup layer. In addition, it is preferable to provide a thin makeup layer on the skin to improve transparency, but when the aspect ratio of the primary particles of hexagonal boron nitride is set to a too large value to improve transparency, an oil absorption value increases, which tends to make it difficult to mix a hexagonal boron nitride powder with other ingredients when preparing cosmetics. Furthermore, a surface treatment or ingredient adjustment may be additionally required, which may impair the original touch feel of hexagonal boron nitride or complicate the manufacturing process of cosmetics. There is room for improvement from the viewpoint of exhibiting spreadability, transparency, and glossiness when used as cosmetics in a well-balanced manner.

An object of the present disclosure is to provide a hexagonal boron nitride powder for cosmetics which is excellent in spreadability and allows a makeup layer to have excellent transparency and glossiness when used as a cosmetic.

### Solution to Problem

One aspect of the present disclosure provides a hexagonal boron nitride powder for cosmetics containing primary particles of hexagonal boron nitride, in which an aspect ratio of the primary particles is 25 or less, and an oil absorption value is 50 to 90 mL/100 g.

The above-mentioned hexagonal boron nitride powder for cosmetics can be suitably used as a raw material for cosmetics because the aspect ratio of the primary particles is within a predetermined range, and the oil absorption value is a specific value. When the hexagonal boron nitride powder is used as a cosmetic, it can exhibit excellent spreadability and can form a makeup layer that can exhibit excellent transparency and glossiness.

In the above-mentioned hexagonal boron nitride powder, a BET specific surface area may be 1.5 to 5.0 m²/g.

In the above-mentioned hexagonal boron nitride powder, a tap density may be 0.35 g/cm³ or less.

In the above-mentioned hexagonal boron nitride powder, a total oxygen amount may be 0.01% to 0.20% by mass.

Another aspect of the present disclosure provides a cosmetic containing the above-mentioned hexagonal boron nitride powder for cosmetics.

The above-mentioned cosmetic contains the above-mentioned hexagonal boron nitride powder, and thus is excellent in spreadability and allows a makeup layer formed using the cosmetic to have excellent transparency and glossiness.

### Advantageous Effects of Invention

According to the present disclosure, it is possible to provide a hexagonal boron nitride powder for cosmetics which is excellent in spreadability and allows a makeup layer to have excellent transparency and glossiness when used as a cosmetic.

### DESCRIPTION OF EMBODIMENTS

The embodiments of the present disclosure will be described below. However, the following embodiments are examples for describing the present disclosure and are not intended to limit the present disclosure to the following contents.

For materials exemplified in the present specification, one type can be used alone, or two or more types can be used in combination, unless explicitly described otherwise. When a plurality of substances corresponding to each of components in a composition are present, the content of each of the components in the composition means the total amount of the plurality of substances present in the composition unless explicitly described otherwise.

A hexagonal boron nitride powder for cosmetics of one embodiment contains primary particles of hexagonal boron nitride, in which an aspect ratio of the primary particles is 25 or less, and an oil absorption value is 50 to 90 mL/100 g.

The shape of the primary particles of hexagonal boron nitride is preferably a scale shape to improve smoothness, spreadability, and concealability. The upper limit value of the aspect ratio of the primary particles of hexagonal boron nitride may be 22 or less, 20 or less, 19 or less, or 18 or less, for example. When the upper limit value of the aspect ratio is within the above-mentioned range, the primary particles have an appropriate thickness, thereby preventing cracking and the like of the primary particles, which makes it possible to minimize an increase in the amount of eluted boron. In addition, light reflection is prevented, which makes it possible to impart an appropriate degree of transparency. The lower limit value of the aspect ratio of the above-mentioned primary particles may be 5 or more, 7 or more, 10 or more, 12 or more, or 15 or more, for example. When the lower limit value of the aspect ratio is within the above-mentioned range, and when used as an extender pigment in a cosmetic, the spreadability of the obtained cosmetic obtained can be further improved. Furthermore, when the lower limit value of the aspect ratio is within the above-mentioned range, and when the hexagonal boron nitride powder is used as an extender pigment for cosmetics, the obtained cosmetic can exhibit excellent concealability (covering up power). The aspect ratio of the primary particles of hexagonal boron nitride may be adjusted within the above-mentioned range, and may be 5 to 22, 10 to 22, or 15 to 22, for example.

The aspect ratio of the primary particles is expressed by the ratio ((major axis)/(minor axis)) of the longest part (major axis) to the shortest part (minor axis) of the particle. In the case of hexagonal boron nitride, since the primary particles are scale-shaped particles, the thickness of the scale-shaped particles is the shortest part (minor axis) of the particle. In other words, the “aspect ratio of the primary particles” in the present specification means a value obtained by calculating the above-mentioned ratio from actual measurement results by actually measuring a particle major axis from an electron microscope image of the primary particles of hexagonal boron nitride, and actually measuring a particle thickness from a cross-sectional photographic image. That is, the aspect ratio of the primary particles of hexagonal boron nitride is a value expressed by (major axis)/(thickness) of the primary particles of hexagonal boron nitride.

When measuring the aspect ratio of scale-shaped particles such as hexagonal boron nitride, for example, in a method that analyzes a particle image taken by an electron microscope as it is, errors are likely to occur (for example, when the primary particles are inclined, an error occurs on the short side (corresponding to a particle thickness and a particle minor axis)), which makes accurate measurement difficult. Therefore, the aspect ratio of hexagonal boron nitride in the present specification is calculated using the particle major axis and the particle minor axis of the primary particles of hexagonal boron nitride which are obtained by measurement in accordance with the method described below. First, regarding the particle major axis of the primary particles of hexagonal boron nitride, the hexagonal boron nitride powder is taken with a scanning electron microscope, and the obtained particle image is imported into image analysis software to measure the long side of the primary particles from the obtained photograph. Next, the particle minor axis of the primary particles of hexagonal boron nitride is measured. First, 3 g of a boron nitride powder is formed into a disk shape (diameter: 30 mmφ) under a pressure of 5 MPa using a press forming machine. After embedding the formed body obtained using resin, a cross-sectional milling process is performed in a direction parallel to the direction in which pressure has been applied to prepare a sample in which the cross-sections of the boron nitride particles are exposed. Because the primary particles of boron nitride are oriented in one direction by press forming, measurement errors on the short side due to the inclination of the primary particles can be minimized. This cross section is taken with a scanning electron microscope, and the obtained particle image is imported into image analysis software to measure the short side from the obtained photograph. For both the particle major axis and the particle minor axis, 100 arbitrarily selected primary particles are measured, and the arithmetic mean value thereof is adopted.

The upper limit value of the oil absorption value of the hexagonal boron nitride powder may be 88 mL/100 g or less, 86 mL/100 g or less, 85 mL/100 g or less, or 80 mL/100 g or less, for example. When the hexagonal boron nitride powder in which the upper limit value of the oil absorption value is within the above-mentioned range is used as an extender pigment for cosmetics, because dispersibility with oily materials can be maintained, it is not required to perform a surface treatment or the like to mix with an oily material when formulating, which makes easy formulation possible while maintaining the original touch feel of hexagonal boron nitride. The lower limit value of the oil absorption value of the hexagonal boron nitride powder may be 55 mL/100 g or more, 60 mL/100 g or more, 65 mL/100 g or more, 70 mL/100 g or more, or 75 mL/100 g or more, for example. When the lower limit value of the oil absorption value is within the above-mentioned range, it becomes possible to reduce the affinity for sebum and prevent makeup from breaking down by sebum. For example, the oil absorption value of the hexagonal boron nitride powder can be controlled by adjusting the BET specific surface area of the primary particles, and can be controlled by adjusting conditions such as a heating temperature at the time of producing the hexagonal boron nitride powder. The oil absorption value of the hexagonal boron nitride powder may be adjusted within the above-mentioned range, and may be 50 to 88 mL/100 g, 60 to 88 mL/100 g, or 70 to 86 mL/100 g, for example.

The “oil absorption value” in the present specification is a value measured in accordance with the method described in “Test Methods For Pigments—Part 13: Oil Absorption—Section 1: Refined Linseed Oil Method” of JIS K 5101-13-1:2004. The oil absorption value corresponds to an oil amount when a sample comes into a paste state when an oil is added dropwise onto the sample. For example, in the case of a sample with high lipophilicity, the oil absorption value is small because a paste state is obtained with a small amount of an oil, whereas in the case of a sample with high hydrophilicity (a sample with low affinity for oil), the oil absorption value is large because a large amount of an oil is required to obtain a paste state.

The lower limit value of the BET specific surface area of the above-mentioned hexagonal boron nitride powder may be 1.5 m²/g or more, 1.8 m²/g or more, 1.9 m²/g or more, 2.0 m²/g or more, 2.3 m²/g or more, or 2.5 m²/g or more, for example. When the lower limit value of the BET specific surface area is within the above-mentioned range, it becomes possible to prevent excessively glowing look of a makeup layer when the hexagonal boron nitride powder is used as a raw material for cosmetics. The upper limit value of the BET specific surface area of the hexagonal boron nitride powder may be 5.0 m²/g or less, 4.0 m²/g or less, or 3.0 m²/g or less, for example. By setting the upper limit value of the BET specific surface area within the above-mentioned range, more preferable glossiness can be imparted to the makeup layer by an appropriate degree of glowing while reducing the amount of eluted boron of the hexagonal boron nitride powder. The BET specific surface area may be adjusted within the above-mentioned range, and may be 1.5 to 5.0 m²/g, for example. The BET specific surface area of hexagonal boron nitride can be controlled by adjusting conditions such as a heating temperature at the time of producing the hexagonal boron nitride powder, for example.

In the present specification, the “BET specific surface area” is a value measured by a single point BET method using nitrogen gas in accordance with the method described in “Determination of the specific surface area of powders (solids) by gas adsorption-BET method” of JIS Z 8830:2013.

The upper limit value of the tap density of the hexagonal boron nitride powder may be 0.35 g/cm³ or less, 0.30 g/cm³ or less, 0.25 g/cm³ or less, or 0.23 g/cm³ or less, for example. When the upper limit value of the tap density is within the above-mentioned range, because the density is low, there is less resistance at the time of application, and hexagonal boron nitride can be spread with light force while being thinly fitted to the skin, which makes it possible to impart better transparency to the makeup layer. The lower limit value of the tap density of the above-mentioned hexagonal boron nitride powder is usually 0.02 g/cm³ or more, or 0.05 g/cm³ or more, but may be 0.08 g/cm³ or more, 0.10 g/cm³ or more, 0.10 g/cm³ or more, 0.15 g/cm³ or more, or 0.20 g/cm³ or more, for example. When the lower limit value of the tap density is within the above-mentioned range, handleability at the time of production and mixing with an oily material for formulation become easier. The tap density of the hexagonal boron nitride powder may be adjusted within the above-mentioned range, and may be 0.02 to 0.35 g/cm³, 0.05 to 0.35 g/cm³, or 0.20 to 0.35 g/cm³, for example.

The tap density in the present specification means a value obtained in accordance with “Test Methods For Bulk Density Of Fine Ceramic Powder” of JIS R 1628:1997. A commercially available device can be used for the measurement. Specifically, measurement is performed under the conditions described in Examples.

The upper limit value of the total oxygen amount of the above-mentioned hexagonal boron nitride powder may be 0.20% by mass or less, 0.15% by mass or less, 0.12% by mass or less, or 0.10% by mass or less, for example. When the upper limit value of the total oxygen amount is within the above-mentioned range, the adhesion and the aggregation of hexagonal boron nitride particles with each other can be reduced, which can further improve spreadability. The lower limit value of the total oxygen amount of the above-mentioned hexagonal boron nitride powder is 0.01% by mass or more, 0.02% by mass or more, 0.03% by mass or more, 0.04% by mass or more, or 0.05% by mass or more, for example. When the lower limit value of the total oxygen amount is within the above-mentioned range, the dispersibility in polar solvents, and the like can be further improved. Therefore, when cosmetics are prepared using the hexagonal boron nitride powder as an extender pigment, mixing with other pigments or the like is easy, which makes it possible to smoothly produce cosmetics. The total oxygen amount may be adjusted within the above-mentioned range, and may be 0.01% to 0.20% by mass, or 0.01% to 0.10% by mass, for example. The total oxygen amount can be controlled by adjusting conditions such as a heating temperature at the time of producing the hexagonal boron nitride powder, for example.

The “total oxygen amount” in the present specification means the total oxygen amount in the hexagonal boron nitride powder. The total oxygen amount can be obtained by the following procedure. The oxygen amount and the nitrogen amount in the hexagonal boron nitride powder is analyzed using an oxygen and nitrogen analyzer. The temperature of a measurement sample is raised in a helium gas atmosphere from 20° C. to about 2500° C., that is, to a temperature higher than the reaction decomposition temperature of boron nitride. Oxygen that is desorbed as the temperature rises is detected. At the beginning of temperature rise, oxygen bonded to the surface of the hexagonal boron nitride powder is desorbed. The surface oxygen amount can be obtained by quantitatively determining the desorbed oxygen. Thereafter, when the temperature reaches around 1400° C., hexagonal boron nitride begins to decompose. The start of decomposition of hexagonal boron nitride can be identified when nitrogen begins to be detected. When hexagonal boron nitride begins to decompose, the oxygen inside the hexagonal boron nitride particles is desorbed. By quantitatively determining the desorbed oxygen amount at this stage, the internal oxygen amount can be obtained. The total value of the surface oxygen amount obtained in this manner and the internal oxygen amount is the total oxygen amount.

In the hexagonal boron nitride powder, the lower limit value of the average particle size may be 4 μm or more, 5 μm or more, 7 μm or more, or 8 μm or more, for example. When the lower limit value of the average particle size is within the above-mentioned range, and when the hexagonal boron nitride powder is used as an extender pigment for cosmetics, the spreadability of the obtained cosmetic can be further improved. The upper limit value of the above-mentioned average particle size may be 19 μm or less, 18 μm or less, 17 μm or less, or 16 μm or less, for example. By setting the upper limit value of the average particle size within the above-mentioned range, excessively glowing look can be prevented. The average particle size may be adjusted within the above-mentioned range, and may be 4 to 19 μm, for example. The average particle size can be controlled by adjusting conditions such as a heating temperature at the time of producing the hexagonal boron nitride powder, for example.

The average particle size in the present specification means a 50% cumulative size (median size) in a volume-based cumulative particle size distribution. The phrase “50% cumulative size in a volume-based cumulative particle size distribution” in the present specification means a particle size (D50) when a cumulative value in a volume-based cumulative particle size distribution is 50% when the particle size distribution of the hexagonal boron nitride powder is measured by a laser diffraction/scattering method. Regarding the laser diffraction/scattering method, the measurement is performed in accordance with the method described in “Particle Size Analysis—Laser Diffraction Methods” of JIS Z 8825:2013. For the measurement, a laser diffraction/scattering particle size distribution measurement device or the like can be used. As the laser diffraction/scattering particle size distribution measurement device, it is possible to use “LS-13 320” (product name) manufactured by Beckman Coulter, Inc., or the like, for example.

The amount of eluted boron is sufficiently reduced in the hexagonal boron nitride powder. The amount of eluted boron in the hexagonal boron nitride powder can be set to 20 mass ppm or less, 15 mass ppm or less, 10 mass ppm or less, 8 mass ppm or less, or 6 mass ppm or less, for example. Irritation to the skin can be reduced by reducing the amount of eluted boron in the hexagonal boron nitride powder, making it more useful as an extender pigment used in cosmetics.

The “the amount of eluted boron” in the present specification means a value measured in accordance with the description of the Japanese Standards of Quasi-drug Ingredients 2006.

The above-mentioned hexagonal boron nitride powder for cosmetics can be suitably used as an extender pigment and can be said to be a raw material for cosmetics. Therefore, the above-mentioned hexagonal boron nitride powder can be called an extender pigment for cosmetics. In addition, the present disclosure can also provide a cosmetic containing the above-mentioned hexagonal boron nitride powder.

Examples of cosmetics include foundations (powder foundation, liquid foundation, cream foundation), face powders, makeup products for highlighting a focal point (point makeup products), eyeshadows, eyeliners, nail polishes, lipsticks, blushes, and mascaras. Among these, the hexagonal boron nitride powder is particularly well suited for foundations and eyeshadows. The content of the hexagonal boron nitride powder in the cosmetic is 0.1% to 70% by mass, for example. Cosmetics can be produced by known methods. A method for producing cosmetics includes a step of blending and mixing the hexagonal boron nitride powder and other raw materials, for example.

The above-mentioned hexagonal boron nitride powder for cosmetics can be produced by the following method, for example. An example of the method for producing the hexagonal boron nitride powder for cosmetics includes: a step of firing a raw material composition containing a boron-containing compound including boric acid and a nitrogen-containing compound including melamine at 600° C. to 1300° C. in an atmosphere containing at least one of an inert gas and an ammonia gas to obtain a calcined product containing at least one selected from the group consisting of low crystallinity boron nitride and amorphous boron nitride (hereinafter also referred to as a calcination step); a step of firing a mixed powder containing the calcined product and an assistant at a temperature of 1500° C. to 1750° C. in an atmosphere containing at least one of an inert gas and an ammonia gas to obtain a fired product (hereinafter also referred to as a firing step); a step of pulverizing, cleaning, and drying the above-mentioned fired product to obtain a dry powder (hereinafter also referred to as a purification step); and a step of heat-treating the above-mentioned dry powder at a temperature of 1900° C. or higher in an atmosphere containing at least one of an inert gas and an ammonia gas (annealing step).

The above-mentioned firing step may be repeated multiple times (which are respectively referred to as a first firing step, a second firing step, and the like in this order hereinafter). When the firing step is repeated multiple times, a fired product obtained in each of the firing steps may be pulverized. By pulverizing the fired product, it is possible to sufficiently consume melamine and the like in the raw material composition in the firing steps after the second firing step. In addition, a pulverization step may include cleaning and drying the powder obtained by pulverization to obtain a dry powder.

The boron-containing compound is a compound having a boron atom as a constituent element. In addition to boric acid, the boron-containing compound may further include boron oxide, borax, and the like, for example. The nitrogen-containing compound is a compound having a nitrogen atom as a constituent element, and may be an organic compound. In addition to melamine, the nitrogen-containing compound may further include dicyandiamide, urea, and the like, for example. The raw material composition may contain components other than the above-mentioned compounds. For example, carbonate such as lithium carbonate and sodium carbonate may be contained as a calcination assistant. Furthermore, a reducing substance such as carbon may be contained.

In the above-mentioned raw material composition, the blending ratio of the boron-containing compound and the nitrogen-containing compound may be adjusted based on the molar ratio of boron atoms to nitrogen atoms. For example, blending may be such that boron atoms:nitrogen atoms=2:8 to 8:2, or blending may be such that boron atoms:nitrogen atoms=2.5:7.5 to 7.5:2.5.

In the calcination step, the above-mentioned raw material composition is calcined using, for example, an electric furnace to obtain the calcined product. The calcination step is performed in an atmosphere containing at least one of an inert gas and an ammonia gas. Examples of the inert gases include nitrogen gas and rare gases. The rare gas may be helium gas or argon gas, for example. The calcination step may be performed in a mixed gas atmosphere in which an inert gas and an ammonia gas are mixed. The calcination temperature may be 600° C. to 1300° C., 800° C. to 1200° C., or 900° C. to 1100° C., for example. The calcination time may be 0.5 to 5.0 hours, or 1.0 to 4.0 hours, for example.

The calcined product obtained by calcination contains at least one selected from the group consisting of low crystallinity boron nitride and amorphous boron nitride, and may further contain hexagonal boron nitride. In the calcination step, the reaction of boron nitride is advanced at a temperature lower than that in the firing step to be described later. By lowering the calcination temperature, grain growth can be minimized, which makes it possible to reduce the average grain size of the finally obtained hexagonal boron nitride powder. Furthermore, by lowering the calcination temperature, grain growth can be minimized, which makes it possible to increase the BET specific surface area of the hexagonal boron nitride powder.

Subsequently, in the firing step, an assistant is mixed with the calcined product obtained as described above to prepare a mixed powder, which is then fired. In the firing step, the generation and the crystallization of boron nitride are advanced while sufficiently consuming the raw material composition in the presence of the assistant. Accordingly, hexagonal boron nitride can be formed with increased crystallinity of boron nitride contained in the calcined product. In the above-mentioned mixed powder, boric acid may be further blended.

Examples of assistants include borate salts such as sodium borate, and carbonates such as sodium carbonate, calcium carbonate, and lithium carbonate. The assistant preferably includes sodium carbonate. The blending amount of the assistant with respect to 100 parts by mass of the calcined product containing boron nitride is equal to or more than 2 parts by mass and less than 20 parts by mass, but may be 3 to 10 parts by mass, or 3 to 7 parts by mass, for example.

In the firing step, the mixed powder is fired using, for example, an electric furnace to obtain a fired product. The firing step is performed in an atmosphere containing at least one of an inert gas and an ammonia gas. Examples of the inert gases include nitrogen gas and rare gases. The rare gas may be helium gas or argon gas, for example. The firing step may be performed in a mixed gas atmosphere containing an inert gas and an ammonia gas.

The firing temperature is 1500° C. to 1750° C. This firing temperature may be 1550° C. to 1850° C., or 1600° C. to 1750° C., for example. The firing time may be 0.5 to 5 hours, or 1 to 4 hours, for example.

In the present specification, the firing time, the heating time, the calcination time, or the like means a time (retention time) for maintaining a predetermined temperature after a temperature of the surrounding environment of an object attains the predetermined temperature.

By setting the firing temperature to be relatively high, the raw material composition is consumed, and amorphous carbon, graphite, or the like generated by the reaction of the raw material composition is consumed, which makes it possible to sufficiently advance the generation and the crystallization of hexagonal boron nitride. By reducing a carbon-containing raw material such as melamine in the raw material composition, the quality of the obtained hexagonal boron nitride powder can be further improved. A similar tendency occurs when the firing time is increased. Meanwhile, when the firing temperature becomes too high, crystal growth of hexagonal boron nitride tends to progress too much, making fine pulverization difficult. A similar tendency occurs when the firing time is too long.

In pulverization of the fired product obtained in the firing step, a pulverizing device may be used, for example. As the pulverizing device, for example, an impact pulverizer or the like may be used. As the impact pulverizer, it is possible to suitably use an impact screen-type pulverizer in which the particle size of a pulverized product can be adjusted by a screen, for example. The aperture of the screen may be 0.1 to 1 mm, or 1 to 3 mm, for example.

In the pulverization step, the above-mentioned fired product is pulverized to adjust the particle size. By adjusting the particle size, the efficiency of the subsequent annealing step can be improved. The pulverized product obtained by pulverizing the fired product may contain impurities other than hexagonal boron nitride. Therefore, a treatment (purification treatment) of reducing the impurities may be performed before the annealing step. Examples of the impurities include remaining raw materials and assistants, and water-soluble boron compounds. The purification treatment reduces the amount of such impurities by cleaning or the like, for example. After cleaning, solid-liquid separation is performed, and drying is performed to obtain a dry powder. By performing the pulverization step and the purification treatment before the annealing step, a powder or dry powder in which the content of the assistant or the like has been reduced than that of the above-mentioned fired product is prepared, and by annealing the powder or dry powder, the oxygen amount can be further reduced while minimizing grain growth.

Examples of cleaning liquids used for cleaning include an aqueous solution containing water and an acidic substance, an organic solvent, and a mixed liquid of an organic solvent and water. From the viewpoint of avoiding secondary incorporation of impurities, water having an electrical conductivity of 1 mS/m or less may be used. Examples of the aqueous solutions containing an acidic substance include inorganic acids such as hydrochloric acid and nitric acid. Examples of the organic solvents include water-soluble organic solvents such as methanol, ethanol, propanol, isopropyl alcohol, and acetone. There are no particular limitations on a cleaning method, and for example, the pulverized product may be cleaned by being immersed in a cleaning liquid and stirred, or the pulverized product may be cleaned by spraying a cleaning liquid thereon.

After the cleaning is completed, the cleaning liquid may be subjected to solid-liquid separation using decantation, a suction filter, a pressure filter, a rotary filter, a sedimentation separator, or a combination of these. The separated solid content may be dried with a general dryer to obtain a dry powder. Examples of the dryers include tray dryers, fluidized bed dryers, spray dryers, rotary dryers, belt dryers, and combinations of these. For example, classification by a sieve may be performed after drying to remove coarse particles.

In the annealing step, the pulverized product or dry powder of the fired product is heat-treated using an electric furnace, for example. The annealing step is performed in an atmosphere containing at least one of an inert gas and an ammonia gas. Examples of the inert gases include nitrogen gas and rare gases. The rare gas may be helium gas or argon gas, for example. The calcination step may be performed in a mixed gas atmosphere containing an inert gas and an ammonia gas. The temperature of the heat treatment in the annealing step is 1900° C. or higher, but from the viewpoint of sufficiently reducing the oxygen amount, the temperature may be 1950° C. or higher, or may be 2000° C. or higher. By performing the annealing step, oxygen present as a functional group or the like on the surface of the particles can be scattered, which makes it possible to reduce the oxygen amount.

From the viewpoint of minimizing particle growth, the temperature of the heat treatment in the annealing step may be 2200° C. or lower, or 2100° C. or lower. The heating time in the annealing step may be 0.5 to 5.0 hours, or 1.0 to 4.0 hours, for example, from the viewpoint of sufficiently reducing the oxygen amount and minimizing particle growth.

Hereinbefore, although some embodiments have been described, the explanations for the common constitutions can be applied to each other. Furthermore, the present disclosure is not limited to the above-mentioned embodiments.

### EXAMPLES

The contents of the present disclosure will be described in more detail with reference to examples and comparative examples, but the present disclosure is not limited to the following examples.

### Example 1

### [Production of Hexagonal Boron Nitride Powder]

### <Calcination Step>

100.0 g of a boric acid powder (purity: 99.8% by mass or more, manufactured by Kanto Chemical Co., Inc.) and 90.0 g of a melamine powder (purity: 99.0% by mass or more, manufactured by FUJIFILM Wako Pure Chemical Corporation) were mixed for 10 minutes using an alumina mortar to obtain a mixed raw material. The mixed raw material after drying was put in a container made of hexagonal boron nitride and disposed in an electric furnace. While circulating nitrogen gas in the electric furnace, the temperature was raised from room temperature to 1000° C. at a rate of 10° C./minute. After retaining at 1000° C. for 2 hours, heating was stopped, and natural cooling was carried out. The electric furnace was opened when the temperature became 100° C. or lower. In this manner, a low crystallinity calcined product containing boron nitride was obtained.

### <Firing Step>

To 100.0 g of the calcined product, 5.0 g of sodium carbonate (purity: 99.5% by mass or more) was added as an assistant, and mixed for 10 minutes using an alumina mortar. The mixture was disposed in the above-mentioned electric furnace. While circulating nitrogen gas in the electric furnace, the temperature was raised from room temperature to 1600° C. at a rate of 10° C./minute. After retaining the firing temperature at 1600° C. for 4 hours, heating was stopped, and natural cooling was carried out. The electric furnace was opened when the temperature became 100° C. or lower. The obtained fired product was recovered and pulverized in an alumina mortar for 3 minutes to obtain a coarse powder containing hexagonal boron nitride.

### <Purification Step>

In order to reduce the impurities contained in the above-mentioned coarse powder, 30 g of the coarse powder was charged to 500 g of dilute nitric acid (nitric acid concentration: 5% by mass) and stirred at room temperature for 60 minutes. After stirring, the solid-liquid separation was performed by suction filtration, and cleaning was performed until the filtrate became neutral by replacing water (water having an electrical conductivity of 1 mS/m). After cleaning, drying was performed at 120° C. for 3 hours using a dryer to obtain a dry powder.

### <Annealing Step>

The dry powder was disposed in the above-mentioned electric furnace. While circulating nitrogen gas in the electric furnace, the temperature was raised from room temperature to 2000° C. at a rate of 10° C./minute. After retaining at 2000° C. for 4 hours, heating was stopped, and natural cooling was carried out. The electric furnace was opened when the temperature became 100° C. or lower.

### <Deagglomeration Step>

The obtained fired product was recovered and pulverized in an alumina mortar for 3 minutes. From the obtained dry powder, a coarse powder was removed using an ultrasonic vibrating sieve (KFS-10000, manufactured by KOWA KOGYOSHO CO., LTD., aperture 250 μm) to obtain a hexagonal boron nitride powder of Example 1.

### [Measurement of Physical Properties of Hexagonal Boron Nitride Powder]

For the hexagonal boron nitride powder prepared in Example 1, the aspect ratio of the primary particles, the oil absorption value, the BET specific surface area of the primary particles, the tap density, and the total oxygen amount were evaluated by the methods described below. Table 1 shows the results.

### <Aspect Ratio>

The aspect ratio of the primary particles of hexagonal boron nitride was determined by calculating the ratio of a major axis to a minor axis (major axis/minor axis) using the particle major axis and particle minor axis of the primary particles of the hexagonal boron nitride obtained by the method described below. Regarding the particle major axis, the hexagonal boron nitride powder instead of the formed body was placed on a carbon tape on an electron microscope sample stage, the sample from which excess powder was removed by air spray or the like was taken with a scanning electron microscope (manufactured by JEOL Ltd., trade name: JSM-6010LA), the obtained particle image was loaded using image analysis software (manufactured by Mountech Co., Ltd., trade name: Mac-View), and the long side (corresponding to the particle major axis) was calculated from the obtained photograph and combined with the minor axis to calculate the aspect ratio (major axis/minor axis). Regarding the particle minor axis, first, 3 g of the hexagonal boron nitride powder was formed into a disk shape (diameter: 30 mmφ) under a pressure of 5 MPa using a press forming machine (manufactured by Rigaku Corporation, trade name: BRE-32), and the obtained formed body was embedded using a resin (manufactured by Gatan, Inc., trade name: G2 Epoxy). Subsequently, a cross-sectional milling process was performed in a direction parallel to the direction in which pressure had been applied to prepare a sample in which the cross-sections of the hexagonal boron nitride particles were exposed. This cross section was taken with a scanning electron microscope (manufactured by JEOL Ltd., trade name: JSM-6010LA), the obtained particle image was loaded using image analysis software (manufactured by Mountech Co., Ltd., trade name: Mac-View), and the short side (corresponding to the particle thickness, the particle minor axis) of the rectangular particles was measured from the obtained photograph. For both the particle major axis and the particle minor axis, 100 arbitrarily selected primary particles were measured, and the arithmetic mean value thereof was adopted.

### <Oil Absorption Value>

The oil absorption value of the hexagonal boron nitride powder was measured in accordance with the method described in “Test Methods For Pigments—Part 13: Oil Absorption—Section 1: Refined Linseed Oil Method” of JIS K 5101-13-1:2004.

### <BET Specific Surface Area of Primary Particles>

The BET specific surface area of the primary particles of hexagonal boron nitride was measured by a single point BET method using nitrogen gas in accordance with the method described in “Determination of the specific surface area of powders (solids) by gas adsorption—BET method” of JIS Z 8830:2013.

### <Tap Density>

Regarding the tap density, in accordance with “Test Methods For Bulk Density Of Fine Ceramic Powder” of JIS R 1628:1997, a 100 cm³ special container was filled with an object to be measured, and after tapping under the conditions of a tapping time: 180 seconds, the number of times of tapping: 180 times, and a tap lift: 18 mm, the bulk density was measured, and the obtained value was defined as the tap density.

### <Total Oxygen Amount>

The total oxygen amount of the primary particles of hexagonal boron nitride was measured using a simultaneous oxygen/nitrogen analyzer (manufactured by HORIBA, Ltd., device name: EMGA-920). Specifically, the measurement was performed while heating the hexagonal boron nitride powder from 20° C. to 2500° C. in a helium atmosphere.

### [Evaluation of Hexagonal Boron Nitride Powder as Raw Material for Cosmetics]

The hexagonal boron nitride powder prepared in Example 1 was evaluated for spreadability when used as a cosmetic, and for transparency and glossiness of a makeup layer composed of the above-mentioned cosmetic by the method described below.

### <Evaluation of Spreadability>

0.2 g of the hexagonal boron nitride powder was put on one end of the artificial skin (length×width=10 mm×50 mm) in a width of 10 mm. The hexagonal boron nitride powder was spread in a longitudinal direction using a spatula to apply the hexagonal boron nitride powder onto the surface of the artificial skin. Image analysis was performed using commercially available image analysis software (WinROOF) to obtained the ratio of the application area of the hexagonal boron nitride powder to the total area of the artificial skin. The larger this area ratio is, the better the spreadability is. Based on the obtained results, the spreadability was evaluated according to the following criteria. Table 1 shows the evaluation results.
A: the ratio of the above-mentioned application area was 95% or more.
B: the ratio of the above-mentioned application area was equal to or more than 80% and less than 95%.
C: the ratio of the above-mentioned application area was equal to or more than 70% and less than 80%.
D: the ratio of the above-mentioned application area was equal to or more than 60% and less than 70%.
E: the ratio of the above-mentioned application area was equal to or more than 40% and less than 60%.
F: the ratio of the above-mentioned application area was less than 40%.

### <Evaluation of Transparency and Glossiness of Makeup Layer>

Sensory evaluation was performed on the transparency and the glossiness of the makeup layer. The evaluation was performed by ten randomly selected expert panelists. The evaluation items were the transparency and the glossiness of the makeup layer. The evaluation was performed on a 5-point scale as follows: the transparency and the glossiness of the makeup layer (reference makeup layer) formed using the hexagonal boron nitride powder obtained in Example 2 were respectively set to “3,” the case superior than the reference makeup layer was set to “4,” the case more superior than the reference makeup layer was set to “5,” the case inferior to the reference makeup layer was set to “2,” and the case even inferior to the reference makeup layer was set to “1.” The arithmetic mean value of the evaluation results of the ten expert panelists was defined as the evaluation result of the makeup layer to be evaluated, and determination was made based on the following criteria. Table 1 shows the results. Regarding the “transparency,” a difference from the reference makeup layer was evaluated using a measure of whether the finished makeup layer felt like it was integrated with the skin without feeling thick. Regarding the “glossiness,” a difference from the reference makeup layer was evaluated using a measure of whether the finished makeup layer looked glossy.
A: the above-mentioned evaluation result was 4.5 or higher.
B: the above-mentioned evaluation result was equal to or higher than 3.5 and lower than 4.5.
C: the above-mentioned evaluation result was equal to or higher than 2.5 and lower than 3.5.
D: the above-mentioned evaluation result was equal to or higher than 1.5 and lower than 2.5.
E: the above-mentioned evaluation result was lower than 1.5.

### Comparative Example 1

A hexagonal boron nitride powder was prepared in the same manner as in Example 1 except that the firing temperature in the firing step was 1800° C. Then, in the same manner as in Example 1, each of measurement and evaluation of the hexagonal boron nitride powder was performed. The results were as shown in Table 1.

### Example 2

A hexagonal boron nitride powder was prepared in the same manner as in Example 1 except that the firing temperature in the annealing step was 1800° C. Then, in the same manner as in Example 1, each of measurement and evaluation of the hexagonal boron nitride powder was performed. The results were as shown in Table 1.

### Comparative Example 2

A hexagonal boron nitride powder was prepared in the same manner as in Example 1 except that the annealing step was not performed. Then, in the same manner as in Example 1, each of measurement and evaluation of the hexagonal boron nitride powder was performed. The results were as shown in Table 1.

### Comparative Example 3

A hexagonal boron nitride powder was prepared in the same manner as in Example 1 except that the addition amount of sodium carbonate in the firing step was 20 g. Then, in the same manner as in Example 1, each of measurement and evaluation of the hexagonal boron nitride powder was performed. The results were as shown in Table 1.

TABLE 1

| | | Example | Comparative | Example | Comparative | Comparative |
|---|---|---|---|---|---|---|
| | | 1 | Example 1 | 2 | Example 2 | Example 3 |
| Hexagonal | Aspect ratio | 20 | 18 | 18 | 20 | 26 |
| boron | Oil absorption | 78 | 105 | 85 | 120 | 70 |
| nitride | value [mL/100 | | | | | |
| powder | g] | | | | | |
| | Specific surface | 2.4 | 1.8 | 2.8 | 5.2 | 1.9 |
| | area [m²/g] | | | | | |
| | Tap density | 0.20 | 0.40 | 0.30 | 0.40 | 0.18 |
| | [g/cm³] | | | | | |
| | Total oxygen | 0.02 | 0.40 | 0.08 | 1.00 | 0.04 |
| | amount [% by | | | | | |
| | mass] | | | | | |
| Evaluation | Spreadability | A | C | C | E | B |
| | Transparency | B | D | C | E | E |
| | Glossiness | A | C | C | D | D |

### INDUSTRIAL APPLICABILITY

According to the present disclosure, an object thereof is to provide a hexagonal boron nitride powder for cosmetics which is excellent in spreadability and allows a makeup layer to have excellent transparency and glossiness when used as a cosmetic.

1. A hexagonal boron nitride powder for cosmetics comprising
primary particles of hexagonal boron nitride,
wherein an aspect ratio of the primary particles is 25 or less, and
an oil absorption value is 50 to 90 mL/100 g.

2. The hexagonal boron nitride powder for cosmetics according to claim 1, wherein a BET specific surface area is 1.5 to 5.0 m²/g.

3. The hexagonal boron nitride powder for cosmetics according to claim 1, wherein a tap density is 0.35 g/cm³ or less.

4. The hexagonal boron nitride powder for cosmetics according to claim 1, wherein a total oxygen amount is 0.01% to 0.20% by mass.

5. A cosmetic comprising the hexagonal boron nitride powder for cosmetics according to claim 1.
